(19) United States
(12) Patent Application Publication
(10) Pub. No.: US 2019/0206518 A1
(43) Pub. Date: Jul. 4, 2019

(54) METHOD OF PROVIDING ONLINE MENTAL-HEALTH CARE

(71) Applicant: Pearson Education, Inc., New York, NY (US)

(72) Inventor: Prashant Banerjee, Bengaluru (IN)

(21) Appl. No.: 15/858,500

(22) Filed: Dec. 29, 2017

(51) Int. Cl. G16H 10/20; G16H 50/20; G16H 80/00; G16H 10/60

(52) U.S. Cl. CPC G16H 10/20; G16H 10/60; G16H 80/00; G16H 50/20

(57) ABSTRACT

Users may download a user application and therapists may download a therapist application to their respective client devices to create a computer network designed for therapists to deliver mental-health care to users that may be geographically separated from the therapists. The applications may gather information about users and therapists to assist in matching users to an appropriate therapist, select appropriate assessment tools for the user and generate an intervention plan. A machine learning algorithm may generate the intervention plan based on the user information (possibly including social media user information), responses to the screening questionnaire, results of assessment tools and/or analysis of one or more recorded video sessions.

### FIELD OF THE INVENTION

This disclosure relates to systems and methods for a therapist using a therapist application running on a therapist client device to provide online mental-health care to a patient/client/user using a user application running on a user client device from any geographical distance.

### SUMMARY OF THE INVENTION

The present invention provides systems and methods comprising one or more computer software programs running on one or more server hardware computing devices or client hardware computing devices (such as a user client device and a therapist client device), communicatively coupled to a network, and each comprising at least one processor executing specific computer-executable instructions within a memory. In addition, the system and methods use one or more databases.

A method is disclosed for providing mobile mental-health care to a user using a user mobile mental-health application (user application) running on a user client device. A therapist may be providing the mobile mental-health care using a therapist mobile mental-health application (therapist application) running on a therapist client device. The method allows the user running the user application on the user client device to be geographically separated from the therapist running the therapist application on the therapist client device.

The therapist may request, using the therapist client device, a therapist application to be downloaded from a publicly available database to the therapist client device. Upon receiving the request from the therapist client device for the therapist application, the publicly available database running on one or more hardware servers, may download the therapist application to the therapist client device. The therapist client device may then run the therapist application on the therapist client device.

The user may request, using the user client device, a user application to be downloaded from the publicly available database to the user client device. Upon receiving the request from the user client device for the user application, the publicly available database running on one or more hardware servers, may download the user application to the user client device. The user client device may then run the user application on the user client device. In this manner, any number of different therapists and users may participate in the overall system that allows therapists to remotely provide mental-health services to clients.

The therapist application running on the therapist client device may collect therapist information from the therapist. The therapist information may comprise contact information, areas of expertise in treating mental-health problems and availability. User applications from past patients/clients of the therapist may also collect therapist information comprising reviews, comments and/or scores for the therapist from the past patients/clients. The therapist information may be stored in a private database. The system may verify the identity and the qualifications of the therapist prior to allowing the therapist to join the network and/or run the therapist application.

The user application running on the user client device may collect user information from the user. The user information supplied by the user may comprise contact information, symptoms, account information for social media platforms used and availability. The user application and system may collect social media user information from time to time from the social media platforms used by the user. The user information may be stored in a private database. The system may verify the identity of the user and confirm the user information supplied by the user matches the social media user information. Serious mismatches (as may be defined as desired) between the user information supplied by the user and the social media user information may prevent the user from jointing the network or the mismatches may be provided to a therapist once the user has selected the therapist.

The user may use the user application to research different mental-health issues. If the user decides the user needs the help of a therapist, the user may use the user application to request mental-health treatment from a therapist. The user application and/or backend system may match the user information with therapist information from a plurality of therapists. The user application and/or backend system may use artificial intelligence (AI) or a machine learning algorithm to match the user information to one or more of the therapists in the plurality of therapists based on any desired factors or number of factors. As non-limiting examples, the Al machine learning algorithm may match symptoms of the user to areas of treatment offered by each therapist and/or match the availability of the user with the availability of each therapist to determine one or more therapists to recommend and display to the user on the user client device running the user application. The system may read past reviews, comments and/or scores from the determined therapists and display this information to the user as the user is selecting a therapist from the list of determined therapist(s).

The user may select one of the listed and displayed therapists using the user application. Upon receiving by the user application the selection of the therapist from the one or more displayed therapists, the user application may administer a screening questionnaire to the user through the user application. The screening questionnaire may be a default screening questionnaire or the selected therapist may have selected a particular screening questionnaire from a list of available screening questionnaires listed on the therapist application or the therapist may have uploaded a screening questionnaire from the therapist application to be stored in a database and used for each user that selects the therapist.

The therapist application may display the user supplied information, social media user information and/or the answers/results of the screening questionnaire (or preferably reports generated therefrom) to the therapist on the therapist client device. In preferred embodiments, the information is analyzed and critical information is highlighted and/or presented as a warning or alert in the therapist application. As a non-limiting example, if the user information does not match information gained from a social media platform used by the user, this information may be highlighted in the therapist application when presented to the therapist. Such mismatched information may be an indication of dishonesty or other mental-health issues of the user.

The therapist may be given an option to reject the user as a client or patient or accept the user as a client or patient. The therapist may reject the client for any reason, such as if the therapist thinks the therapist does not have the proper mental-health skill set or experience to help the user or if the therapist does not believe they have the availability necessary to help the user. If rejected, the user may be given a chance to select another therapist from an updated list that does not include the therapist that rejected the user. If the therapist does accept the user as a client/patient, the therapist and user may schedule a video session. The user application and therapist application may enable and record the video session between the user and the therapist. The system may store the video session in a database, where the user and therapist may see the video session at a later date using the user application or the therapist application.

The system may select and suggest one or more assessment tools to the therapist, based on the user information, collected social media user information from one or more social media platforms, the screening questionnaire and/or one or more video sessions. These sources of information may be analyzed and/or parsed for keywords to determine one or more suggested assessment tools. Specifically, one or more keywords found in any of the user information sources may trigger a particular assessment tool to be suggested to the therapist. In addition, or alternatively, the user sources of information may be analyzed by AI or a machine learning algorithm to determine a different one or more suggested assessment tools. These suggested assessment tools, determined by any desired method(s), may be displayed to the therapist through the therapist application.

The therapist may select one or more of the suggested assessment tools using the therapist application. Upon receiving a selection of the one or more assessment tools from the therapist application, the user application may administer the one or more selected assessment tools to the user through the user application running on the user client device.

The user application or the therapist application may use the responses from the user to score each of the assessment tools and generate a score, i.e., report. The therapist application may display the score/report of the user taking each assessment tool to the therapist using the therapist application. The score/report of the user taking the one or more assessment tools may be stored in a private database for later use as a baseline for the user at the time the user took the assessment tools. The score/report may be available to the user or therapist at a later time for reference.

The therapist application or backend system may use the user information (user supplied and/or social media user information collected from social media platforms), screening questionnaire, score(s) from assessment tools and/or an analysis of video sessions as input to an AI or a machine learning algorithm to determine an intervention plan (treatment plan) for the user. The therapist, through the therapist application, may display the intervention plan to the user, through the user application. The user application may transmit an accept or a reject for the intervention plan to the therapist application. The therapist may coordinate and administer the intervention plan with the user via the therapist application and the user application.

In some embodiments, from time to time (which may be entered or selected by the therapist) or upon a specific request by the therapist, the system may collect or download information regarding the user from one or more social media platforms. The system may use account login information previously provided by the user. The system may analyze images, video, audio and/or text found on the social media platforms related to the user using any desired method. As a non-limiting example, the system may parse the social media user information for keywords that are known to be used by those having particular mental-health issues (such as those ready to commit violence against themselves or against others). As another non-limiting example, the system may use AI or a machine learning algorithm to match the social media user information against patterns of social media user information known in the past to be from those ready to commit violence against themselves or against others. If the system determines the user has used a keyword or is exhibiting a pattern of behavior known to be problematic, the system may immediately send a warning to the therapist on the therapist application and/or possibly warn others that the user has given permission to receive this information.

The above features and advantages of the present invention will be better understood from the following detailed description taken in conjunction with the accompanying drawings.

### BRIEF DESCRIPTION OF THE DRAWINGS

FIG. 1 illustrates a system level block diagram for a non-limiting example distributed computing environment that may be used to practice the invention.

FIG. 2 illustrates a system level block diagram for a computer system that may be used to practice the invention. The illustrated computer system may be representative of the clients in FIG. 1, user client device, therapist client device in FIG. 3 or various hardware servers used in practicing the invention as part of the system/backend of the invention depicted in FIGS. 4 and 5.

FIG. 3 is a block diagram of a system that may be used to download a user application to a user client device and download a therapist application to a therapist client device.

FIG. 4 is a block diagram illustrating the system after the user client device received the user application and therapist client device received the therapist application. This embodiment also illustrates that the system/back end of the invention is connected to one or more social medial platforms, thereby allowing the system/back end to receive social media user information from said social media platforms.

FIG. 5 illustrates that the system/back end may comprise several machine learning algorithms that may be used to match a user to a therapist, select assessment tools for a user and/or select an intervention plan for the user. The system/back end may also comprise a database, wherein the database may further comprise a public database and a private database.

FIGS. 6-9 illustrate a possible method that may be used by the present invention for a therapist using a therapist application running on a therapist client device to provide online mental-health care to a patient/client/user using a user application running on a user client device from any geographical distance.

### DETAILED DESCRIPTION

The present inventions will now be discussed in detail with regard to the attached drawing figures that were briefly described above. In the following description, numerous specific details are set forth illustrating the Applicant's best mode for practicing the invention and enabling one of ordinary skill in the art to make and use the invention. It will be obvious, however, to one skilled in the art that the present invention may be practiced without many of these specific details. In other instances, well-known machines, structures, and method steps have not been described in particular detail in order to avoid unnecessarily obscuring the present invention. Unless otherwise indicated, like parts and method steps are referred to with like reference numerals.

### Network

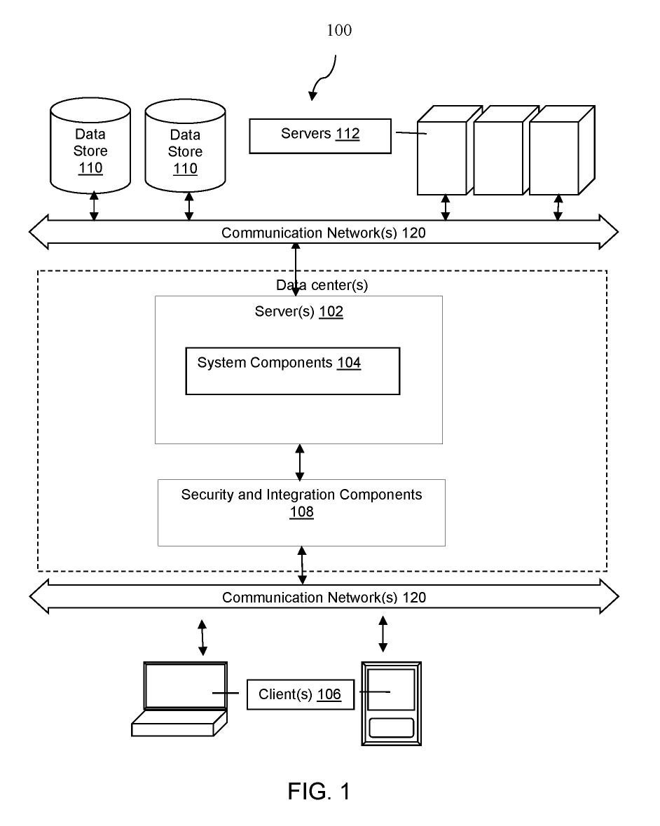

FIG. 1 illustrates a non-limiting example distributed computing environment 100, which includes one or more computer server computing devices 102, one or more client computing devices 106, and other components that may implement certain embodiments and features described herein. Other devices, such as specialized sensor devices, etc., may interact with client 106 and/or server 102. The server 102, client 106, or any other devices may be configured to implement a client-server model or any other distributed computing architecture.

Server 102, client 106, and any other disclosed devices may be communicatively coupled via one or more communication networks 120. Communication network 120 may be any type of network known in the art supporting data communications. As non-limiting examples, network 120 may be a local area network (LAN; e.g., Ethernet, Token-Ring, etc.), a wide-area network (e.g., the Internet), an infrared or wireless network, a public switched telephone networks (PSTNs), a virtual network, etc. Network 120 may use any available protocols, such as (e.g., transmission control protocol/Internet protocol (TCP/IP), systems network architecture (SNA), Internet packet exchange (IPX), Secure Sockets Layer (SSL), Transport Layer Security (TLS), Hypertext Transfer Protocol (HTTP), Secure Hypertext Transfer Protocol (HTTPS), Institute of Electrical and Electronics (IEEE) 802.11 protocol suite or other wireless protocols, and the like.

### Servers/Clients

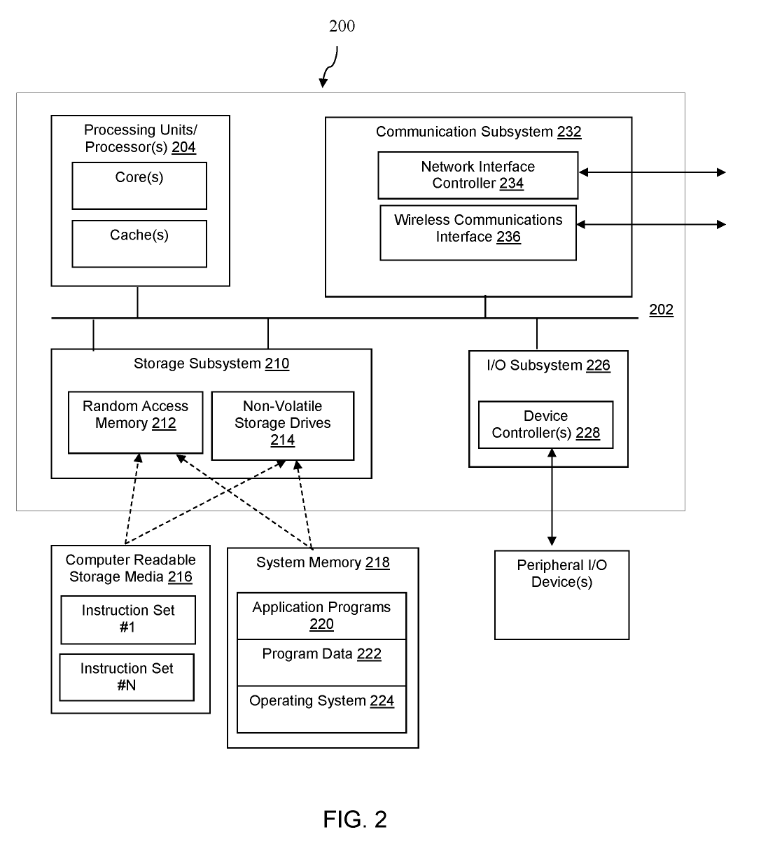

The embodiments shown in FIGS. 1-2 are thus one example of a distributed computing system and is not intended to be limiting. The subsystems and components within the server 102 and client devices 106 may be implemented in hardware, firmware, software, or combinations thereof. Various different subsystems and/or components 104 may be implemented on server 102. Users operating the client devices 106 may initiate one or more client applications to use services provided by these subsystems and components. Various different system configurations are possible in different distributed computing systems 100 and content distribution networks. Server 102 may be configured to run one or more server software applications or services, for example, web-based or cloud-based services, to support content distribution and interaction with client devices 106. Users operating client devices 106 may in turn utilize one or more client applications (e.g., virtual client applications) to interact with server 102 to utilize the services provided by these components. Client devices 106 may be configured to receive and execute client applications over one or more networks 120. Such client applications may be web browser based applications and/or standalone software applications, such as mobile device applications. Client devices 106 may receive client applications from server 102 or from other application providers (e.g., public or private application stores).

### Security

As shown in FIG. 1, various security and integration components 108 may be used to manage communications over network 120 (e.g., a file-based integration scheme or a service-based integration scheme). Security and integration components 108 may implement various security features for data transmission and storage, such as authenticating users or restricting access to unknown or unauthorized users,

As non-limiting examples, these security components 108 may comprise dedicated hardware, specialized networking components, and/or software (e.g., web servers, authentication servers, firewalls, routers, gateways, load balancers, etc.) within one or more data centers in one or more physical location and/or operated by one or more entities, and/or may be operated within a cloud infrastructure.

In various implementations, security and integration components 108 may transmit data between the various devices in the content distribution network 100. Security and integration components 108 also may use secure data transmission protocols and/or encryption (e.g., File Transfer Protocol (FTP), Secure File Transfer Protocol (SFTP), and/or Pretty Good Privacy (PGP) encryption) for data transfers, etc.).

In some embodiments, the security and integration components 108 may implement one or more web services (e.g., cross-domain and/or cross-platform web services) within the content distribution network 100, and may be developed for enterprise use in accordance with various web service standards (e.g., the Web Service Interoperability (WS-I) guidelines). For example, some web services may provide secure connections, authentication, and/or confidentiality throughout the network using technologies such as SSL, TLS, HTTP, HTTPS, WS-Security standard (providing secure SOAP messages using XML encryption), etc. In other examples, the security and integration components 108 may include specialized hardware, network appliances, and the like (e.g., hardware-accelerated SSL and HTTPS), possibly installed and configured between servers 102 and other network components, for providing secure web services, thereby allowing any external devices to communicate directly with the specialized hardware, network appliances, etc.

### Data Stores (Databases)

Computing environment 100 also may include one or more data stores 110, possibly including and/or residing on one or more back-end servers 112, operating in one or more data centers in one or more physical locations, and communicating with one or more other devices within one or more networks 120. In some cases, one or more data stores 110 may reside on a non-transitory storage medium within the server 102. In certain embodiments, data stores 110 and back-end servers 112 may reside in a storage-area network (SAN). Access to the data stores may be limited or denied based on the processes, user credentials, and/or devices attempting to interact with the data store.

### Computer System

With reference now to FIG. 2, a block diagram of an illustrative computer system is shown. The system 200 may correspond to any of the computing devices or servers of the network 100, or any other computing devices described herein. In this example, computer system 200 includes processing units 204 that communicate with a number of peripheral subsystems via a bus subsystem 202. These peripheral subsystems include, for example, a storage subsystem 210, an I/O subsystem 226, and a communications subsystem 232.

### Processors

One or more processing units 204 may be implemented as one or more integrated circuits (e.g., a conventional micro-processor or microcontroller), and controls the operation of computer system 200. These processors may include single core and/or multicore (e.g., quad core, hexa-core, octo-core, ten-core, etc.) processors and processor caches. These processors 204 may execute a variety of resident software processes embodied in program code, and may maintain multiple concurrently executing programs or processes. Processor(s) 204 may also include one or more specialized processors, (e.g., digital signal processors (DSPs), outboard, graphics application-specific, and/or other processors).

### Buses

Bus subsystem 202 provides a mechanism for intended communication between the various components and subsystems of computer system 200. Although bus subsystem 202 is shown schematically as a single bus, alternative embodiments of the bus subsystem may utilize multiple buses. Bus subsystem 202 may include a memory bus, memory controller, peripheral bus, and/or local bus using any of a variety of bus architectures (e.g. Industry Standard Architecture (ISA), Micro Channel Architecture (MCA), Enhanced ISA (EISA), Video Electronics Standards Association (VESA), and/or Peripheral Component Interconnect (PCI) bus, possibly implemented as a Mezzanine bus manufactured to the IEEE P1386.1 standard).

### Input/Output

I/O subsystem 226 may include device controllers 228 for one or more user interface input devices and/or user interface output devices, possibly integrated with the computer system 200 (e.g., integrated audio/video systems, and/or touchscreen displays), or may be separate peripheral devices which are attachable/detachable from the computer system 200. Input may include keyboard or mouse input, audio input (e.g., spoken commands), motion sensing, gesture recognition (e.g., eye gestures), etc.

### Input

As non-limiting examples, input devices may include a keyboard, pointing devices (e.g., mouse, trackball, and associated input), touchpads, touch screens, scroll wheels, click wheels, dials, buttons, switches, keypad, audio input devices, voice command recognition systems, microphones, three dimensional (3D) mice, joysticks, pointing sticks, gamepads, graphic tablets, speakers, digital cameras, digital camcorders, portable media players, webcams, image scanners, fingerprint scanners, barcode readers, 3D scanners, 3D printers, laser rangefinders, eye gaze tracking devices, medical imaging input devices, MIDI keyboards, digital musical instruments, and the like.

### Output

In general, use of the term “output device” is intended to include all possible types of devices and mechanisms for outputting information from computer system 200 to a user or other computer. For example, output devices may include one or more display subsystems and/or display devices that visually convey text, graphics and audio/video information (e.g., cathode ray tube (CRT) displays, flat-panel devices, liquid crystal display (LCD) or plasma display devices, projection devices, touch screens, etc.), and/or non-visual displays such as audio output devices, etc. As non-limiting examples, output devices may include, indicator lights, monitors, printers, speakers, headphones, automotive navigation systems, plotters, voice output devices, modems, etc.

### Memory or Storage Media

Computer system 200 may comprise one or more storage subsystems 210, comprising hardware and software components used for storing data and program instructions, such as system memory 218 and computer-readable storage media 216.

System memory 218 and/or computer-readable storage media 216 may store program instructions that are loadable and executable on processor(s) 204. For example, system memory 218 may load and execute an operating system 224, program data 222, server applications, client applications 220, Internet browsers, mid-tier applications, etc.

System memory 218 may further store data generated during execution of these instructions. System memory 218 may be stored in volatile memory (e.g., random access memory (RAM) 212, including static random access memory (SRAM) or dynamic random access memory (DRAM)). RAM 212 may contain data and/or program modules that are immediately accessible to and/or operated and executed by processing units 204.

System memory 218 may also be stored in non-volatile storage drives 214 (e.g., read-only memory (ROM), flash memory, etc.) For example, a basic input/output system (BIOS), containing the basic routines that help to transfer information between elements within computer system 200 (e.g., during start-up) may typically be stored in the non-volatile storage drives 214.

### Computer Readable Storage Media

Storage subsystem 210 also may include one or more tangible computer-readable storage media 216 for storing the basic programming and data constructs that provide the functionality of some embodiments. For example, storage subsystem 210 may include software, programs, code modules, instructions, etc., that may be executed by a processor 204, in order to provide the functionality described herein. Data generated from the executed software, programs, code, modules, or instructions may be stored within a data storage repository within storage subsystem 210.

Storage subsystem 210 may also include a computer-readable storage media reader connected to computer-readable storage media 216. Computer-readable storage media 216 may contain program code, or portions of program code. Together and, optionally, in combination with system memory 218, computer-readable storage media 216 may comprehensively represent remote, local, fixed, and/or removable storage devices plus storage media for temporarily and/or more permanently containing, storing, transmitting, and retrieving computer-readable information.

Computer-readable storage media 216 may include any appropriate media known or used in the art, including storage media and communication media, such as but not limited to, volatile and non-volatile, removable and non-removable media implemented in any method or technology for storage and/or transmission of information. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media. This can also include nontangible computer-readable media, such as data signals, data transmissions, or any other medium which can be used to transmit the desired information and which can be accessed by computer system 200.

By way of example, computer-readable storage media 216 may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray® disk, or other optical media. Computer-readable storage media 216 may include, but is not limited to, Zip® drives, flash memory cards, universal serial bus (USB) flash drives, secure digital (SD) cards, DVD disks, digital video tape, and the like. Computer-readable storage media 216 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magneto-resistive RAM (MRAM) SSDs, and hybrid SSDs that use a combination of DRAM and flash memory based SSDs. The disk drives and their associated computer-readable media may provide non-volatile storage of computer-readable instructions, data structures, program modules, and other data for computer system 200.

### Communication Interface

Communications subsystem 232 may provide a communication interface from computer system 200 and external computing devices via one or more communication networks, including local area networks (LANs), wide area networks (WANs) (e.g., the Internet), and various wireless telecommunications networks. As illustrated in FIG. 2, the communications subsystem 232 may include, for example, one or more network interface controllers (NICs) 234, such as Ethernet cards, Asynchronous Transfer Mode NICs, Token Ring NICs, and the like, as well as one or more wireless communications interfaces 236, such as wireless network interface controllers (WNICs), wireless network adapters, and the like. Additionally and/or alternatively, the communications subsystem 232 may include one or more modems (telephone, satellite, cable, ISDN), synchronous or asynchronous digital subscriber line (DSL) units, Fire Wire® interfaces, USB® interfaces, and the like. Communications subsystem 236 also may include radio frequency (RF) transceiver components for accessing wireless voice and/or data networks (e.g., using cellular telephone technology, advanced data network technology, such as 3G, 4G or EDGE (enhanced data rates for global evolution), WiFi (IEEE 802.11 family standards, or other mobile communication technologies, or any combination thereof), global positioning system (GPS) receiver components, and/or other components.

### Input Output Streams Etc.

In some embodiments, communications subsystem 232 may also receive input communication in the form of structured and/or unstructured data feeds, event streams, event updates, and the like, on behalf of one or more users who may use or access computer system 200. For example, communications subsystem 232 may be configured to receive data feeds in real-time from users of social networks and/or other communication services, web feeds such as Rich Site Summary (RSS) feeds, and/or real-time updates from one or more third party information sources (e.g., data aggregators). Additionally, communications subsystem 232 may be configured to receive data in the form of continuous data streams, which may include event streams of real-time events and/or event updates (e.g., sensor data applications, financial tickers, network performance measuring tools, clickstream analysis tools, automobile traffic monitoring, etc.). Communications subsystem 232 may output such structured and/or unstructured data feeds, event streams, event updates, and the like to one or more data stores that may be in communication with one or more streaming data source computers coupled to computer system 200.

### Connect Components to System

The various physical components of the communications subsystem 232 may be detachable components coupled to the computer system 200 via a computer network, a FireWire® bus, or the like, and/or may be physically integrated onto a motherboard of the computer system 200. Communications subsystem 232 also may be implemented in whole or in part by software.

### Other Variations

Due to the ever-changing nature of computers and networks, the description of computer system 200 depicted in the figure is intended only as a specific example. Many other configurations having more or fewer components than the system depicted in the figure are possible. For example, customized hardware might also be used and/or particular elements might be implemented in hardware, firmware, software, or a combination. Further, connection to other computing devices, such as network input/output devices, may be employed. Based on the disclosure and teachings provided herein, a person of ordinary skill in the art will appreciate other ways and/or methods to implement the various embodiments.

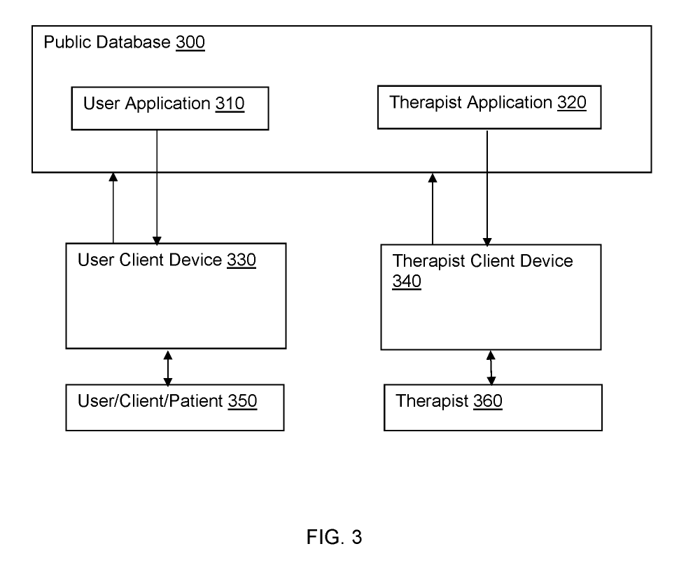

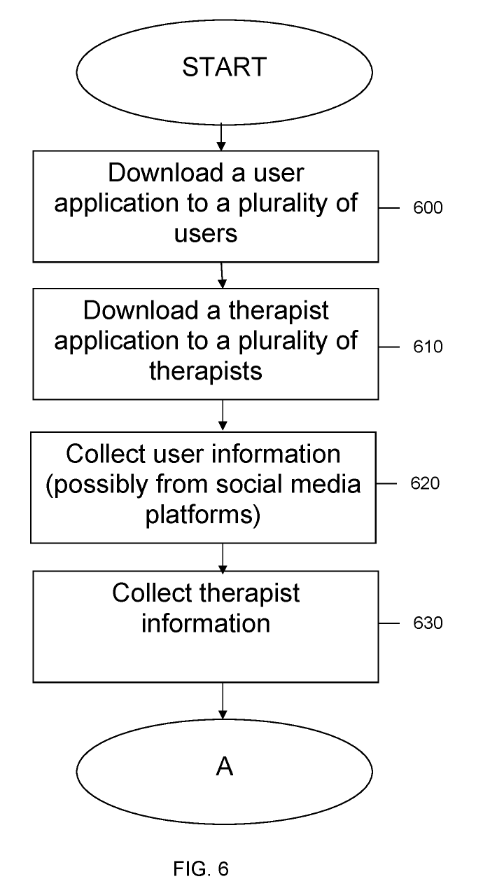

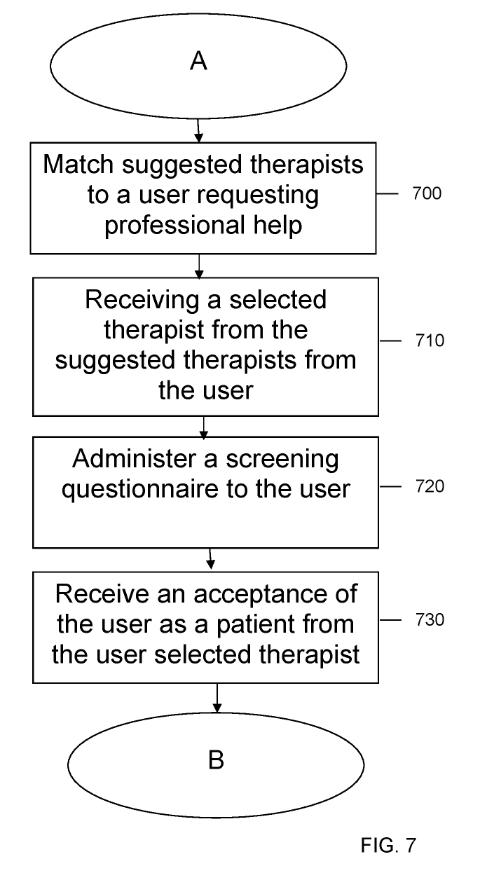

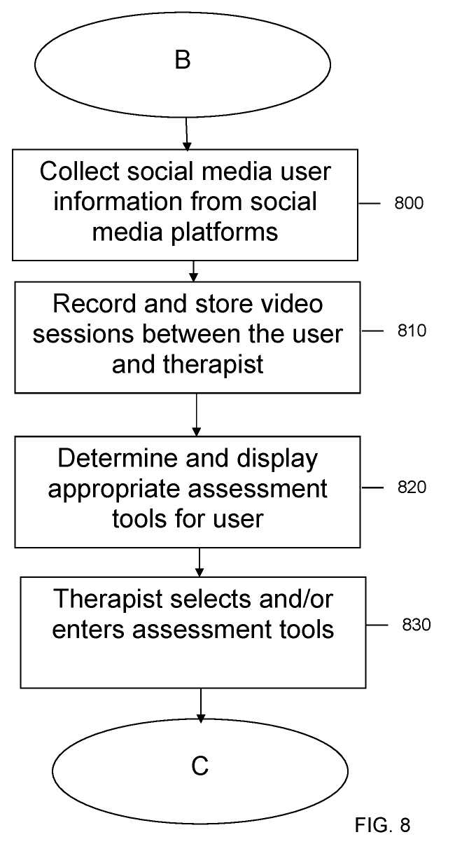

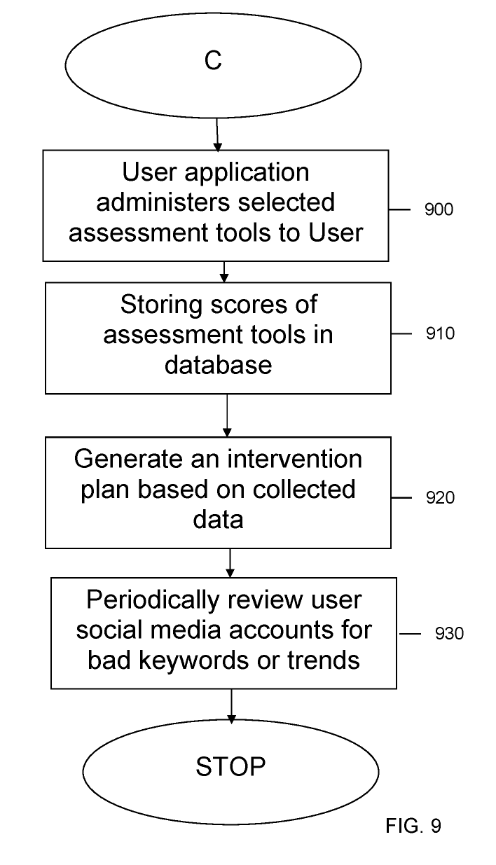

FIG. 3 illustrates a system that may practice a method of the invention which provides mobile mental-health care to a user 350 using a user mobile mental-health application 310 (user application) running on a user client device 330. The user client device 330 may be the client 106 as previously described in FIGS. 1-2. A method of practicing the invention is illustrated in FIGS. 6-9. The user 350 may receive the mobile mental-health care from a therapist 360 using a therapist mobile mental-health application 320 (therapist application) running on a therapist client device 340. The user application 310 and the therapist application 320 may be the same application (although the user application 310 would run on the user client device 330 and the therapist application 320 would run on the therapist client device 340).

If the user application 310 and therapist application 320 are the same, the applications may enable different features, as appropriate, for the user 350 and the therapist 360 as needed for the user 350 to receive mobile mental-health services and for the therapist 360 to provide mobile mental-health services. The user application 310 and therapist application 320 may also be different, with the user application 310 comprising the features needed for the user 350 to receive mobile mental-health services and the therapist application 320 comprising the features needed for the therapist 360 to provide mobile mental-health services.

The user application 310 and therapist application 320 may be stored on a publicly available database 300 so that any number of different users 350 may download a copy of the user application 310 to their user client devices 330 and any number of different therapists 360 may download a copy of the therapist application 320 to their therapist client devices 340.

Thus, upon the publicly available database 300 receiving a request from a therapist client device 340 for a therapist application 320, the publicly available database 300 may download by one or more hardware servers the therapist application 320 from the publicly available database 300 to the therapist client device 340. The therapist 360 may then run the therapist application 320 on the therapist client device 340. This process may be repeated for any number of different therapists 360 to download and run a copy of the therapist application 320 on each of the therapist client device 340. (Step 610)

Each therapist 360 may be a provider of mental-health services and have a professional practice, such as in clinical psychology. The therapists 360 may be part of a preferred service provider network for multiple insurance companies that bring in the users/clients/patients 350, i.e., users 350 of this invention. However, the therapists 360 may have long commutes between the therapist's 360 place of work, the therapist's 360 home and/or various clinics where the therapist 360 works. Due to personal commitments, the therapist 360 may find it increasingly difficult to commute to these places to keep her practice thriving and maintain a work-life balance.

The therapist 360 may download the therapist application 320 from the publicly available database 300 and run the application on the therapist client device 340. The therapist 360 may use the therapist application 320 to sign up, create an account and get access to a digital library of assessment and intervention solutions. The therapist 360 may use these solutions, as examples, on a pay per use or subscription basis. The therapist 360 may also avail themselves of a host of other features built in the therapist application 320, such as video sessions, that take the entire experience of offering psychological services to a whole new level of reach, convenience, affordability and impact.

The therapist 360 may enter contact information, geographical area(s) for desired clients, qualified areas to offer treatment, days and hours of availability, biometric data (fingerprints, face scans, retinal scans, etc.) and/or any other desired information. The therapist application 320 may verify the identity of the therapist 360, preferably via a comparison of collected biometric data for the therapist 360 and known biometric data for the therapist 360, and verify the therapist 360 is qualified to provide mental-heal care in the particular geographic area of the therapist 360, the particular geographic area of the user 350 (client) and/or for the selected areas of treatment. The collected information regarding the therapist 360 may be stored in a database for later use.

The geographic area of the therapist 360 may be determined by reading the location from the therapist client device 340 and/or comparing the IP address of the therapist client device 340 with known locations of IP addresses. The qualifications of the therapist 360 may be determined by downloading public databases comprising a list of qualified therapists 360 within a given geographical region.

Other records may be downloaded to ascertain educational degrees and/or certificates awarded to the therapist 360. If the identity and/or the qualifications of the therapist 360 cannot be verified, the therapist application 320 may prevent the therapist 360 from offering services to users 350 of the user applications 310. (Step 630)

Once the identity and/or qualifications of the therapist 360 have been verified as meeting minimal requirements, the therapist 360 may sign up to use the therapist application 320. The therapist 360 may start to receive new users/clients/patients 350 through the application which may help the therapist 360 create a better work life balance and enable the therapist 360 to expand the therapist's geographical area of practice, such as to an entire country. The therapist 360 may prefer paying for the assessment solutions on a per use basis. Paying for only the test reports the therapist 360 wishes to use for her current clients helps control input costs.

The entire process of test administration, scoring and reporting, combined with progress monitoring on interventions used may be seamless, efficient and cost-effective. The therapist application 320 may empower the therapist 360 and thousands of other mental health professionals around the world to create greater value for their clients, all from the convenience of their homes and offices and sometimes on the go. The therapist application 320 also helps make a real difference in the lives of users/clients/patients 350 who are looking for a fulfilment of their need for psychological help and support. In this manner, any number of different therapists 360 may be verified (identify and qualifications) and allowed to use a copy of the therapist application 320 and participate in providing mental-health care to users 350 using a copy of the user application 310.

A user 350 may desire to receive mental-health care information and/or mental-health care from a therapist 360. The user 350 may desire to use a user application 310 running on the user client device 330 as the provided mental-health care is very private and may be received in any geographical area, even areas that are remote and might not have local therapists 360. The user 350 may discover the user application 310 in a publicly available application database. The user 350 may request a user application 310 be downloaded to the user client device 330 from the publicly available database. The database, upon receiving the user 350 request, may download the user application 310 to the user client device 330. The user 350 may run the user application 310 on the user client device 330 to assist the user 350 in obtaining information and/or mental-health care from a therapist 360. (Step 600)

The user 350 may be any age and experiencing any number of different mental-health issues. As an example, the user 350 may be experiencing depression and anxiety, such as those a student might experience due to academic challenges. However, those with mental-health issues are often reluctant to avail themselves of counselling services as there is a stigma attached to being ‘found out’ in terms of coping with psychological issues. However, the user 350 may use the user application 310 in private to protect the privacy of their personal issues.

The user 350 may sign up using the user application 310 running on the user client device 330. The user 350 may enter contact information, location information, symptoms, areas of desired help, days and hours of availability for treatment, biometric data (fingerprints, face scans, retinal scans, etc.) and/or any other desired information. The user application 310 may verify the identity of the user 350, preferably via a comparison of collected biometric data for the user 350 and known biometric data for the user 350. The collected information regarding the user 350 may be stored in a private database 430 for later use. (Step 620)

Based on the desired areas of help, the user application 310 may prompt the user 350 to read up on different aspects of mental health and well being as well as on specific kinds of psychological disorders such as depression, anxiety, etc. This may be a free section of the user application 310 which is available to all. The user 350 may find this section useful and be interested in exploring further as to how the user application 310 may address the user's 350 psychological and academic challenges. This general mental-health information may be stored in a public database 420 operated by the system 410.

The user 350 may request services from a therapist 360 using the user application 310. Upon receiving a request for mental-health treatment from the user application 310, the system 410 may match the user 350 information with the therapist 360 information from one or more therapists 360 in the plurality of therapists 360. As non-limiting examples, the system 410 may match the user 350 information, i.e., symptoms/issues, availability, desired therapist 360 gender, age, psychological issues, desired therapeutic approach, and location with the therapist 360 information from each therapist 360 registered in the system. The user application 310 may offer paid psychological services based on an aggregator model that matches individuals like the user 350 who are in need of psychological help with the right mental health professional and other services. The system 410 may comprise an AI or machine learning algorithm 520 to match the user 350 with one or more suggested therapists 360. An advantage of the present system is that the distance between the user 350 and the therapist 360 gives the user 350 privacy in sorting through the user's 350 issues and the online services make the distance a non-issue. (Step 700)

The user 350 may receive through the user application 310, one, two or more therapists 360 displayed and listed on the user client device 330. In a preferred embodiment, additional information is listed and associated with each therapist 360. As examples, reviews, ratings, specialties, location, gender, age range, therapeutic approach, education and/or work experience may be displayed for one or more of the therapists 360. In some embodiments, the reviews and ratings may have been entered by past clients of the therapist 360 and stored in a database. Theses reviews and ratings may be displayed by the user application 310 to the user 350 prior to the user 350 selecting a therapist 360. The user 350 may then select the best therapist 360 for the needs of the user 350. (Step 710)

Once the user 350 has selected one of the displayed therapists 360 using the user application 310, the user application 310 may administer a screening questionnaire to the user 350 on the user application 310. The screening questionnaire may be standard and used by all therapists 360 or, alternatively, some or all of the therapists 360 may select or create their own screening questionnaires that are stored in a database. Upon the user 350 selecting a therapist 360, the system may read the screening questionnaire from the database selected by the therapist 360 to be administered to the user 350. This allows each therapist 360 to customize the screening questionnaire so that the questions most important to the therapist 360 in determining if a user 350 is a match for the therapist 360 are asked and answered by the user 350. (Step 720)

The answers from the user 350 to the screening questionnaire may be displayed to the therapist 360 by the therapist application 320 on the therapist client device 340. The therapist application 320 may then display the options of accepting the user 350, rejecting the user 350, or asking additional follow-up questions of the user 350. If the therapist 360 selects asking additional follow-up questions, the additional questions may be displayed to the user 350 using the user application 310.

Once the therapist 360 is satisfied that the therapist 360 would be able to help the user 350 based on the screening questionnaire, the therapist 360 may accept the service request from the user 350 via the therapist application 320. (Step 730) The therapist 360 may then schedule a video session with the user 350 through the therapist application 320 and the user application 310 to have an introductory discussion and agree on the course ahead. The therapist 360 may explain to the user 350 the need to use psychological assessments to first identify the user's 350 issues objectively and scientifically and then plan an intervention path, based on the assessment results. The video interface for the user application 310 and the therapist application 320 allows for a recording of the session. All such recorded video sessions may be automatically stored in one or more secure hardware servers as per patient/client privacy laws in the appropriate country. Both the therapist 360 and the user 350 are preferably able to access these recordings during the course of the professional association (and beyond, as legally appropriate). (Step 810)

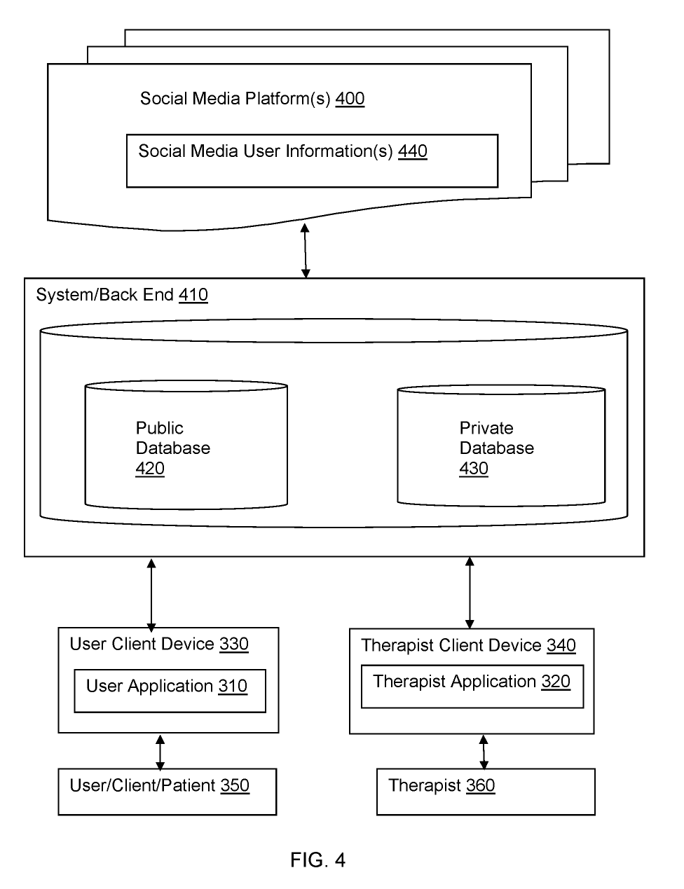

Referring to FIG. 4, the system 410 may collect social media user information 440 from one or more social media platforms 400. Specifically, the system may review any images, video, audio and/or text messages entered by the user 350. In preferred embodiments, this social media user information 440 is periodically collected from the one or more social media platforms 400 and stored in a private database. The system may compare the user 350 supplied information with the social media user information 440. If the user 350 supplied information is different from the social media user information 440, the therapist 360 may be sent a warning along with the mismatching information to assist the therapist 360 in providing mental-health care to the user 350. (Step 800)

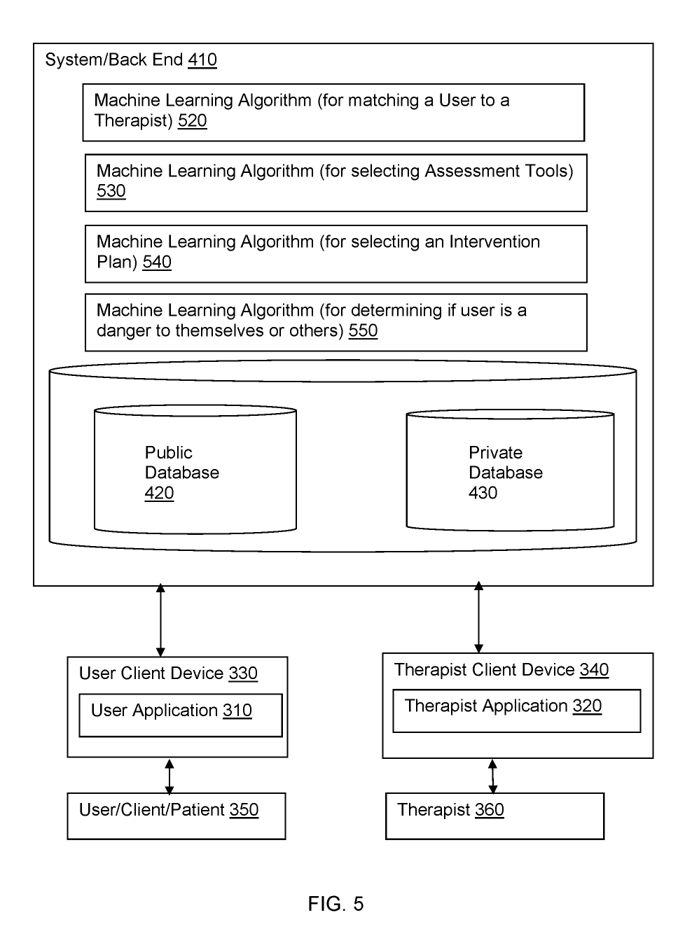

Referring to FIG. 5, the system may comprise a system/back end 410 that analyzes the images, video, audio and/or text messages using any desired technique. As a non-limiting example, the system 410 may search the social media user information 440 for keywords that are known to be used by people who are in danger of harming themselves or others. As another non-limiting example, the system may also use artificial intelligence or a machine learning algorithm 550 to analyze the social media user information 440 to also determine if the user 350 is likely to harm themselves or others. If the system 410 detects that the user 350 is likely to harm themselves or others, a warning message (and possibly the problematic social media user information 440) may be sent to the therapist 360 suggesting that an immediate intervention with the user 350 may be needed.

In another embodiment, the user application 310 could link to one or more of the user's social media accounts such as Facebook® or Twitter®, to recognize suicidal (or homicidal) tendencies through pattern recognition algorithms and alert the therapist 360 (or mental health and/or law enforcement authorities). (Step 930)

The user application 310 may also have a single button SOS feature to enable those who are suicidal to reach out for help. Tapping the button would send an alert to helpline centers and/or the therapist application 320 that could get in touch with the user/client/patient 350 immediately.

In some embodiments, the therapist 360 may enter keywords (possibly names of certain people or other red flag words) that the system may search for in the social media user information 440 retrieved from one or more social media accounts. The therapist 360 may request or even require the user 350 to supply user names, login information, etc. needed to access the user's social media accounts. This information may be used by the system to monitor the social media accounts of the user 350 on one or more social media platforms 400 (such as Facebook®, YouTube®, Twitter®, Instagram®, etc.).

In some embodiments, the therapist application 320 may analyze, such as by using a machine learning algorithm 530, a plurality of keywords entered by the therapist 360 (possibly derived by the therapist 360 based on the user 350 supplied information, the screening questionnaire of the user 350 and/or one or more video session of the user 350 and therapist 360). The analysis may also include the user 350 supplied information and social media user information 440 collected from one or more social media platforms 400. The analysis may be used to list one or more suggested assessment tools to the therapist 360 for use with the user 350. (Step 820) The therapist 360 may select one or more of the suggested assessment tools displayed by the therapist application 320 to be administered to the user 350 by the user application 310 running on the user client device 330. (Step 830)

In some embodiments, the therapist 360 may select one or more of the suggested assessment tools and select or enter one or more assessment tools not in the suggested list, but in a comprehensive list of all assessment tools. This feature allows the therapist 360 to use the therapist's knowledge in selecting the most appropriate battery of assessment tools for the user 350. The therapist application 320 may have one or more fields configured to receive an entered assessment tool.

From the suggested list of assessment tools, the therapist 360 may select a battery of psychological assessments that may include, as non-limiting examples, assessments for emotional difficulties: Beck Depression Inventory (BDI), Beck Anxiety Inventory (BAI) and Beck Suicidal Scale (BSS); assessment for intellectual abilities: Raven's Progressive Matrices; assessment for working memory: Wechsler Memory Scale (WMS) and/or an assessment for learning disability to determine the clinical picture of the user's 350 struggles and the nature of the intervention required. Upon receiving a selection of one or more assessment tools from the therapist application 320, the one or more assessment tools may be administered to the user 350 through the user application 310. (Step 900)

The therapist application 320 may also recommend in a displayed list on the therapist client device 340 one or more intervention solutions, based on one or more of the user supplied information, social media user information 440, screening questionnaire, one or more assessment tools and/or analyzed one or more video sessions, to the therapist 360, such as Cogmed, cognitive behavioral therapy (CBT) strategies, etc. The therapist application 320 may list for each intervention solution an overview of each intervention solution, the intervention solution's clinical application, age range for the intervention solution, etc. As with the assessment tools, the therapist 360 may also select or enter one or more intervention solutions in the therapist application 320 to be administered to the user 350 using the user application 310 running on the user client device 330.

The therapist 360 may administer the digital assessment tools to the user 350 via the user application 310. This provides the therapist 360 a convenient way of administering the tests to the user 350 by using an online assessment and intervention dashboard that the therapist application 320 has given the therapist 360 access to. The therapist 360 is able to assign these assessment tools on the go from the therapist client device 340. Following the administration of the assessment tools, the assessments may be automatically scored and test reports may be generated for the therapist 360 to interpret. The test reports generated may be automatically stored in a secure database running on a hardware server and be assessable to the therapist 360 for reference, during the therapy work with the user 350. (Step 910)

The user application 310 or the therapist application 320 may score the one or more assessment tools and display the score(s) by the therapist application 320 to the user 350 on the user application 310.

The therapist 360 may interpret the test reports and conclude that the user 350 has been experiencing any number of different psychological disorders. As non-limiting examples, the therapist 360 may conclude the user 350 is experiencing clinical levels of depression, borderline anxiety and a significant impairment in the user's 350 working memory capacity. Based on the conclusions drawn, the therapist 360 may conduct additional video sessions with the user 350 to mutually agree on these conclusions and discuss an intervention plan. (Step 920)

Based on the therapist's 360 inputs to the therapist application 320, the therapist application 320 may suggest psychiatrists in the geographic area of the user 350. The user 350 may reach out for, as non-limiting examples, drug therapy, to address the clinical depression. The user 350 may choose to not use these services at the moment but the user 350 may wish to use these services in the future. The same user application 310 may also be used to avail drug therapy with a psychiatrist or psychologist with prescription privileges, via video sessions and online drug prescriptions that could be shared with neighborhood pharmacies listed on the user application 310 and geo tagged for location. If given permission by the user 350, the psychiatrist may be given access to all of the user 350 information stored in the private database.

The user application 310 may also have features to help with drug therapy with possible benefits, such as home delivery of medicines, reminders to restock ahead of the medicines running out, and a complete record of the drug regime that could be shared with the treating doctor and any other mental health professional in future. This improves on the process of exchanging information in situations such as when a change of mental health professional occurs.

Following the assessment process, the therapist 360 may schedule, as a non-limiting example, once-a-week video counselling sessions with the user 350 to work on the user's 350 issues involving, as a non-limiting example, self-esteem (linked to the user's 350 depression). In addition, the therapist 360 may give the user 350 access to a 5-week, 25-day training program, Cogmed, to help improve the user's 350 working memory capacity. The therapist 360 may function as the user's 350 Cogmed coach to monitor the user's 350 progress in the training program and keep the user 350 motivated on the way. The therapist 360 may have access to a separate dashboard to monitor the user's 350 progress and performance on Cogmed that the user 350 can access from within the user application 310. The therapist 360 may also give the user 350 access to the CBT strategies section through the user application 310 that augments insights the user 350 gains during the video counselling sessions.

After each video session, the therapist 360 may record the video, audio and/or written notes from the session on the therapist application 320. The therapist 360 may also record any out-of-session assignment given to the user 350 using the therapist application 320. The user 350 may be able to record the user's 350 experience of the session as well. This feedback may also be shared with the therapist 360 for the user 350 to gain insights on how the user 350 fared in a session with the therapist 360. The user application 310 may send reminders to the user 350 to complete assignments, for upcoming video counselling sessions as well as alerts for missed daily Cogmed sessions.

The user application 310 or the therapist application 320 may integrate an AI or machine learning algorithm 540 and 550 to select and/or predict intervention outcomes for a user 350 who avails themselves to services on the user application 310 and course correction algorithms that may suggest alternative intervention options for the user 350 that are likely to work better based on the treatments/results of past users 350 of the user application 310. The user information, treatments and results for all past users 350 of the user application 310 may be stored in a private database and used as the training data or dataset used by an AI or a machine learning algorithm 550.

With the user's 350 approval, the user application 310 may connect the user 350 with a community of individuals struggling with similar issues. The user application 310 gives the user 350 the option of initially reaching out to these individuals anonymously through the user application 310, to agree and decide on meeting in real-world settings, for group-based support.

The user application 310 may also provide a comparison of the user's 350 pre and post treatment scores on one or more assessment tools, to offer objective data on how the interventions used had made a difference to the user's 350 issues. The user application 310 may remind the user 350 of any future follow-up meeting dates with the therapist 360.

It should be appreciated that the various databases 300, 420, 430 discussed in FIGS. 3-9 may be of the type described as the data store 110 in FIG. 1.

Other embodiments and uses of the above inventions will be apparent to those having ordinary skill in the art upon consideration of the specification and practice of the invention disclosed herein. The specification and examples given should be considered exemplary only, and it is contemplated that the appended claims will cover any other such embodiments or modifications as fall within the true scope of the invention.

The Abstract accompanying this specification is provided to enable the United States Patent and Trademark Office and the public generally to determine quickly from a cursory inspection the nature and gist of the technical disclosure and in no way intended for defining, determining, or limiting the present invention or any of its embodiments.

The invention claimed is:

1. A method of providing mobile mental-health care to a user using a user mobile mental-health application (user application) running on a user client device from a therapist using a therapist mobile mental-health application (therapist application) running on a therapist client device, comprising the steps of:
upon receiving a request from a therapist client device for a therapist application, downloading by one or more hardware servers the therapist application from a publicly available database and running the therapist application on the therapist client device;
upon receiving a request from a user client device for a user application, downloading by the one or more hardware servers the user application from the publicly available database and running the user application on the user client device;
collecting user information from the user application, one or more social media platforms or a private database;
collecting therapist information from a plurality of therapist applications running on a plurality of therapist client devices;
upon receiving a request for mental-health treatment from the user application, matching the user information with the therapist information from one or more therapists in the plurality of therapists;
receiving by the user application reviews and ratings from past patients for each of the one or more matched therapists from a review and rating database;
displaying by the user application for selection by the user the one or more matched therapists, along with the reviews or ratings from the past patients for each of the one or more matched therapist;
upon receiving by the user application a selection of the therapist in the one or more therapists, administering a screening questionnaire to the user by the user application;
displaying by the therapist application results of the screening questionnaire;
upon receiving by the therapist application an acceptance of the user as a patient from the therapist, enabling and recording a video session between the user application and the therapist application;
storing the video session in a private database assessable by the user through the user application and the therapist through the therapist application;
displaying by the therapist application a list of assessment tools for the user based on a plurality of keywords entered by the therapist, a plurality of keywords entered by the user or an analysis of the video session using a machine learning algorithm;
upon receiving a selection of an assessment tool from the therapist application, administering the assessment tool to the user through the user application;
upon the user application or the therapist application scoring the assessment tool, displaying by the therapist application running on the therapist client device a score for the user taking the assessment tool;
storing the score in a private database, assessable by the therapist through the therapist application;
generating an intervention plan for the user using a machine learning algorithm based on the user information, the results of the screening questionnaire, the score of the assessment tool or an analysis of the video session; and
periodically or upon request by the therapist through the therapist application:
downloading information regarding the user from one or more social media platforms,
analyzing the downloaded information regarding the user to determine one or more mental-health problems of the user, and
displaying on the therapist application an alert identifying the user and the one or more mental-health problems of the user.

2. The method of claim 1, wherein the screening questionnaire is selected or uploaded by the therapist prior to the screening questionnaire being administered to the user.

3. The method of claim 1, wherein the displaying by the therapist application the list of assessment tools for the user is based on the plurality of keywords entered by the therapist.

4. The method of claim 1, wherein the displaying by the therapist application the list of assessment tools for the user is based on the plurality of keywords entered by the user.

5. The method of claim 1, wherein the displaying by the therapist application the list of assessment tools for the user is based on an analysis of the video session using a machine learning algorithm.

6. The method of claim 1, wherein the therapist application receives user account access information for one or more social media platforms used by the user from the user through the user application.

7. The method of claim 1, further comprising the steps of:
upon receiving a request for mental health information from the user application, transmitting the requested mental health information from a database to the user application; and
displaying by the user application the requested mental health information on the user client device.

8. A method of providing mobile mental-health care to a user using a user mobile mental-health application (user application) running on a user client device from a therapist using a therapist mobile mental-health application (therapist application) running on a therapist client device, comprising the steps of:
upon receiving a request from a therapist client device for a therapist application, downloading by one or more hardware servers the therapist application from a publicly available database and running the therapist application on the therapist client device;
upon receiving a request from a user client device for a user application, downloading by the one or more hardware servers the user application from the publicly available database and running the user application on the user client device;
collecting user information from one or more social media platforms;
displaying by the user application the collected user information to the user;
receiving edits of the collected user information from the user through the user application;
storing by the user application in a private database the edited collected user information as the user information;
collecting therapist information from a plurality of therapist applications running on a plurality of therapist client devices;
upon receiving a request for mental-health treatment from the user application, matching the user information with the therapist information from one or more therapists in the plurality of therapists;
receiving by the user application reviews and ratings from past patients for each of the one or more matched therapists from a review and rating database;
displaying by the user application for selection by the user the one or more matched therapists, along with the reviews or ratings from the past patients for each of the one or more matched therapist;
upon receiving by the user application a selection of the therapist in the one or more therapists, administering a screening questionnaire to the user by the user application;
displaying by the therapist application results of the screening questionnaire;
upon receiving by the therapist application an acceptance of the user as a patient from the therapist, enabling and recording a video session between the user application and the therapist application;
storing the video session in a private database assessable by the user through the user application and the therapist through the therapist application;
displaying by the therapist application a list of assessment tools for the user based on a plurality of keywords entered by the therapist, a plurality of keywords entered by the user or an analysis of the video session using a machine learning algorithm;
upon receiving a selection of an assessment tool from the therapist application, administering the assessment tool to the user through the user application;
upon the user application or the therapist application scoring the assessment tool, displaying by the therapist application running on the therapist client device a score for the user taking the assessment tool;
storing the score in a private database, assessable by the therapist through the therapist application;
generating an intervention plan for the user using a machine learning algorithm based on the user information, the results of the screening questionnaire, the score of the assessment tool or an analysis of the video session; and
periodically or upon request by the therapist through the therapist application:
downloading information regarding the user from one or more social media platforms,
analyzing the downloaded information regarding the user to determine one or more mental-health problems of the user, and
displaying on the therapist application an alert identifying the user and the one or more mental-health problems of the user.

9. The method of claim 8, wherein the screening questionnaire is selected or uploaded by the therapist prior to the screening questionnaire being administered to the user.

10. The method of claim 8, wherein the displaying by the therapist application the list of assessment tools for the user is based on the plurality of keywords entered by the therapist.

11. The method of claim 8, wherein the displaying by the therapist application the list of assessment tools for the user is based on the plurality of keywords entered by the user.

12. The method of claim 8, wherein the displaying by the therapist application the list of assessment tools for the user is based on an analysis of the video session using a machine learning algorithm.

13. The method of claim 8, wherein the therapist application receives user account access information for one or more social media platforms used by the user from the user through the user application.

14. The method of claim 8, further comprising the steps of:
upon receiving a request for mental health information from the user application, transmitting the requested mental health information from a database to the user application; and
displaying by the user application the requested mental health information on the user client device.

15. A method of providing mobile mental-health care to a user using a user mobile mental-health application (user application) running on a user client device from a therapist using a therapist mobile mental-health application (therapist application) running on a therapist client device, comprising the steps of:
upon receiving a request from a therapist client device for a therapist application, downloading by one or more hardware servers the therapist application from a publicly available database and running the therapist application on the therapist client device;
upon receiving a request from a user client device for a user application, downloading by the one or more hardware servers the user application from the publicly available database and running the user application on the user client device;
collecting by the user application user information from the user;
collecting therapist information from a plurality of therapist applications running on a plurality of therapist client devices;
upon receiving a request for mental-health treatment from the user application, matching the user information with the therapist information from one or more therapists in the plurality of therapists;
receiving by the user application reviews and ratings from past patients for each of the one or more matched therapists from a review and rating database;
displaying by the user application for selection by the user the one or more matched therapists, along with the reviews or ratings from the past patients for each of the one or more matched therapist;
upon receiving by the user application a selection of the therapist in the one or more therapists, administering a screening questionnaire to the user by the user application;
displaying by the therapist application results of the screening questionnaire;
collecting social media user information from one or more social media platforms;
upon determining the user information does not match the social media user information, displaying an alert to the therapist that the user information does not match the social media user information;
upon receiving by the therapist application an acceptance of the user as a patient from the therapist, enabling and recording a video session between the user application and the therapist application;
storing the video session in a private database assessable by the user through the user application and the therapist through the therapist application;
displaying by the therapist application a list of assessment tools for the user based on a plurality of keywords entered by the therapist, a plurality of keywords entered by the user or an analysis of the video session using a machine learning algorithm;
upon receiving a selection of an assessment tool from the therapist application, administering the assessment tool to the user through the user application;
upon the user application or the therapist application scoring the assessment tool, displaying by the therapist application running on the therapist client device a score for the user taking the assessment tool;
storing the score in a private database, assessable by the therapist through the therapist application;
generating an intervention plan for the user using a machine learning algorithm based on the user information, the results of the screening questionnaire, the score of the assessment tool or an analysis of the video session; and
periodically or upon request by the therapist through the therapist application:
downloading information regarding the user from one or more social media platforms,
analyzing the downloaded information regarding the user to determine one or more mental-health problems of the user, and
displaying on the therapist application an alert identifying the user and the one or more mental-health problems of the user.

16. The method of claim 15, wherein the screening questionnaire is selected or uploaded by the therapist prior to the screening questionnaire being administered to the user.

17. The method of claim 15, wherein the displaying by the therapist application the list of assessment tools for the user is based on the plurality of keywords entered by the therapist.

18. The method of claim 15, wherein the displaying by the therapist application the list of assessment tools for the user is based on the plurality of keywords entered by the user.

19. The method of claim 15, wherein the displaying by the therapist application the list of assessment tools for the user is based on an analysis of the video session using a machine learning algorithm.

20. The method of claim 15, wherein the therapist application receives user account access information for one or more social media platforms used by the user from the user through the user application.
